(19) United States
(12) Patent Application Publication
(10) Pub. No.: US 2007/0260307 A1
(43) Pub. Date: Nov. 8, 2007

(54) VISION PROSTHESIS WITH IMPLANTABLE POWER SOURCE

(76) Inventor: Dimitri T. Azar, Brookline, MA (US)

Correspondence Address: FISH & RICHARDSON PC, P.O. BOX 1022, MINNEAPOLIS, MN, 55440-1022, US

(21) Appl. No.: 11/381,772

(22) Filed: May 5, 2006

(51) Int. Cl. A61F 2/16

(52) U.S. Cl. 623/6.22; 623/6.37

(57) ABSTRACT

A vision prosthesis including an intraocular lens having a refractive power that varies in response to a stimulus; and an implantable power source for providing power to an actuator in communication with the intraocular lens for providing the stimulus.

### FIELD OF INVENTION

This invention relates to a vision prostheses, and in particular, to intraocular prostheses.

### BACKGROUND

In the course of daily life, one typically regards objects located at different distances from the eye. To selectively focus on such objects, the focal length of the eye's lens must change. In a healthy eye, this is achieved through the contraction of a ciliary muscle that is mechanically coupled to the lens. To the extent that the ciliary muscle contracts, it deforms the lens. This deformation changes the focal length of the lens. By selectively deforming the lens in this manner, it becomes possible to focus on objects that are at different distances from the eye. This process of selectively focusing on objects at different distances is referred to as “accommodation.”

As a person ages, the lens loses plasticity. As a result, it becomes increasingly difficult to deform the lens sufficiently to focus on objects at different distances. To compensate for this loss of function, it is necessary to provide different optical corrections for focusing on objects at different distances.

One approach to applying different optical corrections is to carry different pairs of glasses and to swap glasses as the need arises. For example, one might carry reading glasses for reading and a separate pair of distance glasses for driving. This is inconvenient both because of the need to carry more than one pair of glasses and because of the need to swap glasses frequently.

Bifocal lenses assist accommodation by integrating two different optical corrections onto the same lens. The lower part of the lens is ground to provide a correction suitable for reading or other close-up work while the remainder of the lens is ground to provide a correction for distance vision. To regard an object, a wearer of a bifocal lens need only maneuver the head so that rays extending between the object-of-regard and the pupil pass through that portion of the bifocal lens having an optical correction appropriate for the range to that object.

The concept of a bifocal lens, in which different optical corrections are integrated into the same lens, has been generalized to include trifocal lenses, in which three different optical corrections are integrated into the same lens, and continuous gradient lenses in which a continuum of optical corrections are integrated into the same lens. However, just as in the case of bifocal lenses, optical correction for different ranges of distance using these multifocal lenses relies extensively on relative motion between the pupil and the lens.

Once a lens is implanted in the eye, the lens and the pupil move together as a unit. Thus, no matter how the patient's head is tilted, rays extending between the object-of-regard and the pupil cannot be made to pass through a selected portion of the implanted lens. As a result, multifocal lenses are generally unsuitable for intraocular implantation because once the lens is implanted into the eye, there can be no longer be relative motion between the lens and the pupil.

A lens suitable for intraocular implantation is therefore generally restricted to being a single focus lens. Such a lens can provide optical correction for only a single range of distances. A patient who has had such a lens implanted into the eye must therefore continue to wear glasses to provide optical corrections for those distances that are not accommodated by the intraocular lens.

### SUMMARY

In one aspect, the invention features a vision prosthesis that includes an intraocular lens having a refractive power that varies in response to a stimulus; and an implantable power source for providing power to an actuator.

Embodiments include those in which the intraocular lens changes refractive power because of a change in index of refraction, a change in the shape of the lens, a change in the relative locations of lens elements relative to each other, or any combination thereof. Some embodiments also include an actuator in communication with the lens to provide the stimulus.

In some embodiments, the power source includes a rechargeable power source. Examples of such power sources include a photovoltaic cell, or a power source configured to be recharged by exposure thereof to an electromagnetic field, for example a magnetic field.

For power sources that include a photovoltaic cell, a light-receiving portion of the cell can be configured for disposition posterior to the iris. In some embodiments, the light-receiving portion is annular. The photovoltaic cell can be configured to be recharged by laser radiation, or by ambient lighting.

In other embodiments, the implantable power source includes a thermoelectric cell.

Additional embodiments include these in which the implantable power source includes a dielectric elastomer coupled to an anatomic structure of the eye for recharging a rechargeable power source.

Certain embodiments feature mechanical systems for capturing mechanical energy for recharging the power source. Examples of such systems include those for capturing kinetic energy associated with movement of an anatomic structure, and those that include a self-winding mechanism configured to capture kinetic energy for recharging the rechargeable power source.

Additional embodiments include those in which the implantable power source includes a mechanical linkage configured for placement between an anatomic structure of the eye and the intraocular lens. Some embodiments include a magnet attached to the intraocular lens, the magnet being responsive to a force applied to the mechanical linkage. One example of a mechanical linkage includes a ring configured for attachment to the ciliary body, and a magnet attached to the ring for exerting force on the magnet attached to the intraocular lens.

These and other features and advantages of the invention will be apparent from the following detailed description and the accompanying figures, in which:

### BRIEF DESCRIPTION OF THE FIGURES

FIG. 1 is a block diagram of the vision prosthesis;

FIGS. 2-5 show the vision prosthesis of FIG. 1 implanted at various locations within the eye;

FIGS. 6, 7A, and 7B show two embodiments of the lens and actuator of FIG. 1;

FIG. 8 shows a feedback mechanism for a rangefinder of the vision prosthesis of FIG. 1;

FIG. 9 shows the vision prosthesis of FIG. 1 mounted on an eyeglass frame; and

FIG. 10 shows a ring mounted to the ciliary body.

### DETAILED DESCRIPTION

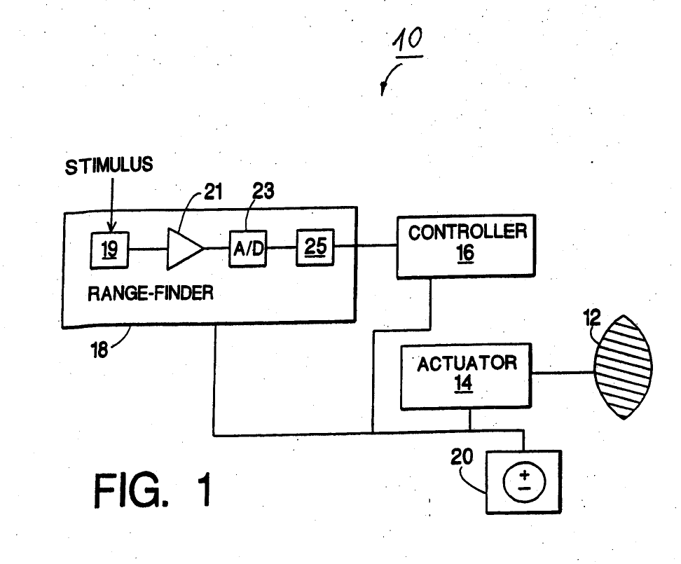

FIG. 1 shows a block diagram of a vision prosthesis 10 having a lens 12 whose refractive power can be made to vary in response to a focusing signal provided to the lens 12 by an actuator 14. In the particular embodiment shown, the refractive power varies because of a change in the index of refraction. In particular, the lens 12 directs light through a nematic liquid-crystal whose index of refraction varies in response to an applied electric field. The actuator 14 includes one or more electrodes in electrical communication with the lens 12. However, the lens 12 can also direct light through a material whose index of refraction varies in response to an applied magnetic field. In this case, the actuator 14 is a magnetic field source, such as a current-carrying coil, in magnetic communication with the lens 12.

Throughout this specification, the terms “lens” and “intraocular lens” refer to the prosthetic lens that is part of the vision prosthesis 10. The lens that is an anatomical structure within the eye is referred to as the “natural lens”.

The nature of the focusing signal provided by the actuator 14 controls the extent to which the refractive power is changed. The actuator 14 generates a focusing signal in response to instructions from a controller 16 in communication with the actuator 14. The controller 16 is typically a microcontroller having instructions encoded therein. These instructions can be implemented as software or firmware. However, the instructions can also be encoded directly in hardware in, for example, an application-specific integrated circuit. The instructions provided to the microcontroller include instructions for receiving, from a rangefinder 18, data indicative of the distance to an object-of-regard, and instructions for processing that data to obtain a focusing signal. The focusing signal alters the lens' refractive power to focus an image of the object-of-regard on the retina.

The rangefinder 18 typically includes a transducer 19 for detecting a stimulus from which a range to an object can be inferred. The signal generated by the transducer 19 often requires amplification before it is of sufficient power to provide to the controller 16. Additionally, the signal may require some preliminary signal conditioning. Accordingly, in addition to a transducer 19, the rangefinder 18 includes an amplifier 21 to amplify the signal, an A/D converter 23 to sample the resultant amplified signal, and a digital signal processor 25 to receive the sampled signal. The output of the digital signal processor 25 is provided to the controller 16.

A power source 20 supplies power to the controller 16, the range finder 18, and the actuator 14. A single power source 20 can provide power to all three components. However, the vision prosthesis 10 can also include a separate power source 20 for any combination of those components that require power.

Lens and Actuator

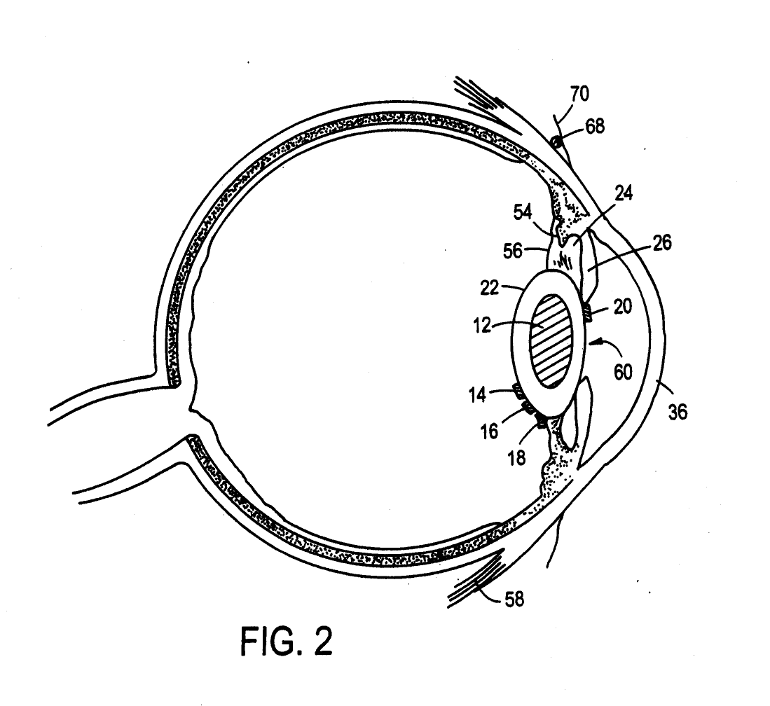

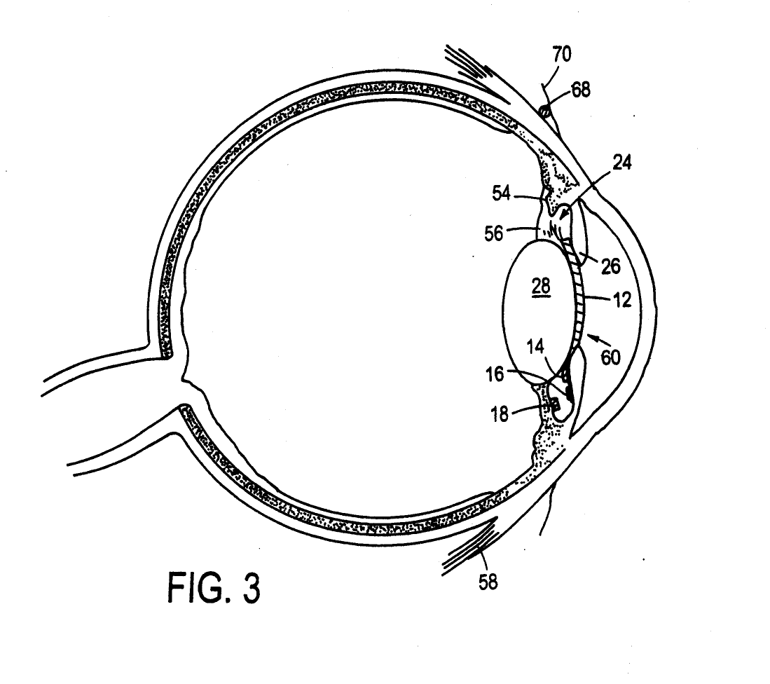

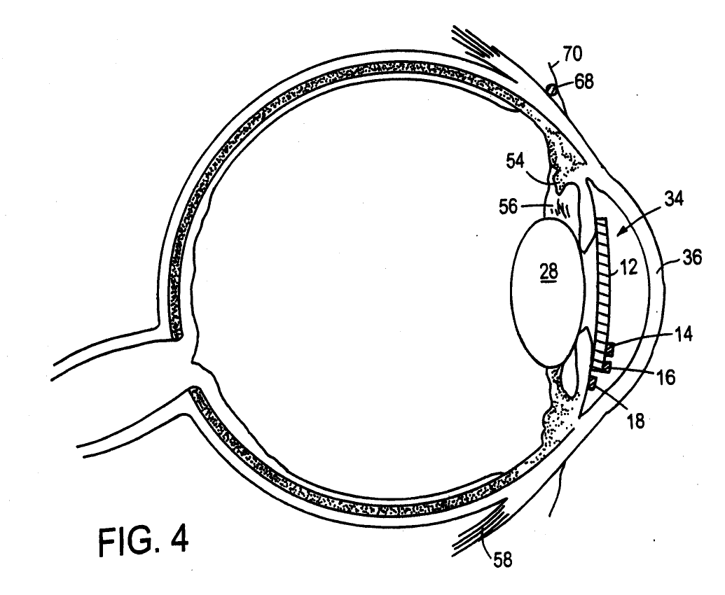

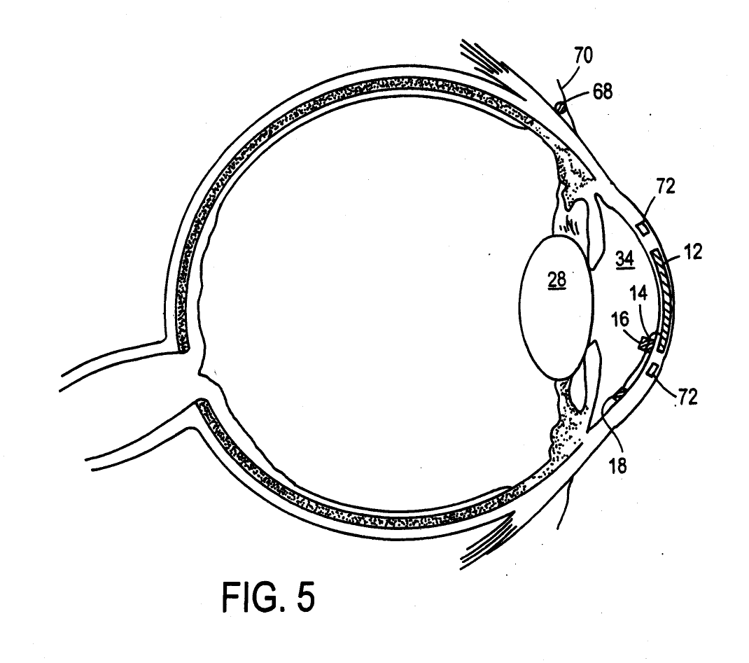

In one embodiment of the vision prosthesis 10, the lens 12 is an intraocular lens. The intraocular lens 12 can be implanted into an aphakic patient, as shown in FIG. 2, in which case it can be implanted into the lens-bag 22 from which the patient's natural lens has been removed. Alternatively, the intraocular lens 12 can be implanted into a phakic patient, in which case it can be implanted into the posterior chamber 24, between the iris 26 and the patient's natural lens 28, as shown in FIG. 3. With the intraocular lens 12 implanted in the posterior chamber 24, the haptic 30 of the lens 12 rests in the sulcus 32. The intraocular lens 12 can also be implanted in the anterior chamber 34, as shown in FIG. 4, or in the cornea 36, as shown in FIG. 5.

Preferably, the lens 12 is a foldable lens having a tendency to spring back to its unfolded position. Such a lens 12 can be inserted through a small incision, maneuvered into the desired location, and released. Once released, the lens 12 springs back to its unfolded position.

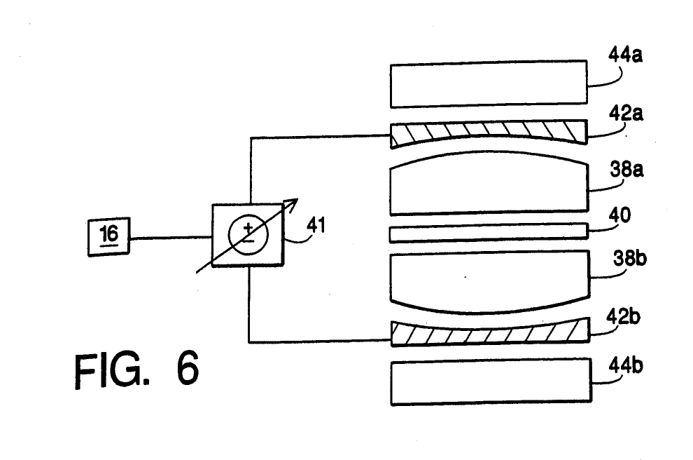

In one embodiment of the lens 12, shown in exploded view in FIG. 6, first and second curved chambers 38a, 38b filled with nematic liquid-crystal are separated by a transparent plate 40. In this embodiment, the actuator 14 includes a variable voltage source 41 connected to two transparent electrodes 42a, 42b disposed on an outer surface of each curved chamber 38a, 38b. The variable voltage source 41 generates a variable voltage in response to instructions from the controller 16. First and second transparent outer layers 44a, 44b cover the first and second electrodes 42a, 42b respectively.

When the variable voltage source 41 applies a voltage, the first and second electrodes 42a, 42b impose an electric field in the nematic liquid-crystal. This electric field tends to reorient the directors of the nematic liquid-crystal, thereby changing its index of refraction. A lens assembly of this type is described fully in U.S. Pat. No. 4,190,330, the contents of which are herein incorporated by reference.

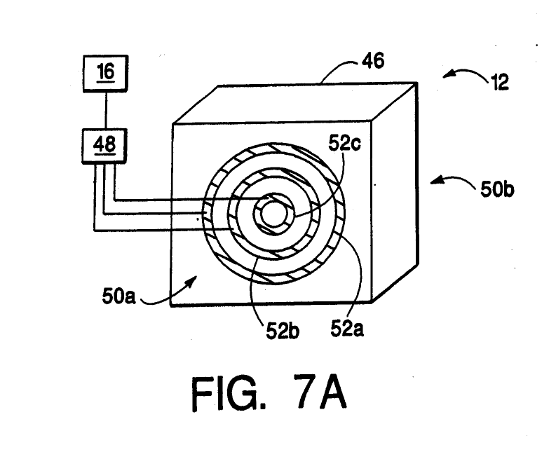

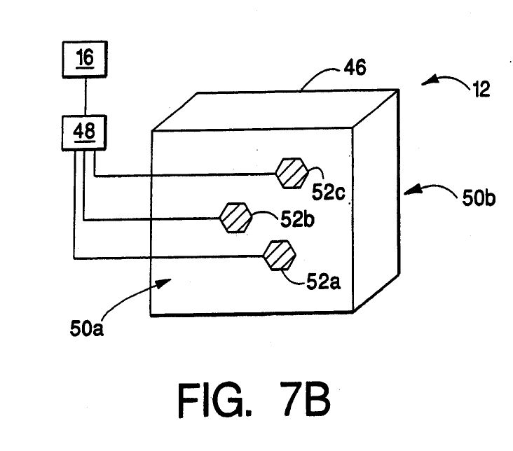

In another embodiment, shown in FIG. 7A, the lens 12 includes a thin chamber 46 filled with nematic liquid-crystal and the actuator 14 includes a variable voltage source 48 and first and second sets 50a, 50b of electrodes 52a-c disposed on opposed planar surfaces of the thin chamber 46. Each of the electrodes 52a-c is individually addressable by the controller 16. A voltage maintained across a electrode 52a form the first set 50a and a corresponding electrode from the second set 50b results in an electric field across a local zone of the nematic liquid-crystal adjacent to those electrodes. This electric field reorients the directors, and hence alters the index of refraction, within that zone. As a result, the index of refraction can be made to vary at different points of the lens 12.

FIG. 7A shows a lens assembly having concentric electrodes 52a-c. A lens assembly of this type is described fully in U.S. Pat. No. 4,466,703, the contents of which are herein incorporated by reference. In this embodiment, the index of refraction can be altered as a function of distance from the center of the lens 12. However, individually addressable electrodes 52a-c can also be arranged in a two-dimensional array on the surface of the lens 12. When this is the case, the index of refraction can be varied as a function of two spatial variables. The grid of electrodes 52a-c can be a polar grid, as shown in FIG. 7A, or a rectilinear grid, as shown in FIG. 7B. The electrodes 52a-c can be distributed uniformly on the grid, or they can be distributed more sparsely in certain regions of the lens 12 and more densely in other regions of the lens 12.

Because of its thin planar structure, a lens 12 of the type shown in FIG. 6 is particularly suitable for implantation in constricted spaces, such as in the posterior chamber 24 of a phakic patient, as shown in FIG. 3.

In another embodiment, the lens 12 includes a chamber filled with a nematic liquid-crystal and the actuator 14 is a current-carrying coil that generates a magnetic field. In this embodiment, the controller 16 causes current to flow in the coil. This current supports a magnetic field that reorients the directors in the nematic liquid-crystal. This results in a change in the liquid crystal's index of refraction.

The extent to which the index of refraction of a nematic liquid crystal can be changed is limited. Once all the directors in the nematic liquid crystal have been polarized, increasing the magnitude of the imposed electric field has no further effect. A nematic liquid crystal in this state is said to be saturated. To change the focal length beyond the point at which the nematic crystal is saturated, a lens 12 can also include one or more lens elements that are moved relative to each other by micromechanical motors.

Alternatively, the lens can have a baseline curvature that and also be filled with nematic crystal. The baseline curvature can be used to perform a gross correction that can be fine-tuned by locally varying the index of refraction of the lens material, or by varying the shape of the lens itself.

In another embodiment, the lens is made up of a multiplicity of lenslets, or lens elements, as shown in FIG. 7B, each of which has its own baseline curvature and each of which is filled with nematic crystal. An individually addressable electrode is then connected to each of the lenslets. In this embodiment, both the lens curvature and the index of refraction can be varied locally and can be varied as a function of two spatial variables.

Rangefinder

In a normal eye, contraction of a ciliary muscle 54 is transmitted to the natural lens 28 by zonules 56 extending between the ciliary muscle 54 and the lens-bag 22. When the object-of-regard is nearby, the ciliary muscle 54 contracts, thereby deforming the natural lens 28 so as to bring an image of the object into focus on the retina. When the object-of-regard is distant, the ciliary muscle 54 relaxes, thereby restoring the natural lens 28 to a shape that brings distant objects into focus on the retina. The activity of the ciliary muscle 54 thus provides an indication of the range to an object-of-regard.

For an intraocular lens 12, the transducer 19 of the rangefinder 18 can be a transducer for detecting contraction of the ciliary muscle 54. In one embodiment, the rangefinder 18 can include a pressure transducer that detects the mechanical activity of the ciliary muscle 54. A pressure transducer coupled to the ciliary muscle 54 can be a piezoelectric device that deforms, and hence generates a voltage, in response to contraction of the ciliary muscle 54. In another embodiment, the transducer can include an electromyograph for detecting electrical activity within the ciliary muscle 54.

As noted above, the activity of the ciliary muscle 54 is transmitted to the natural lens 28 by zonules 56 extending between the ciliary muscle 54 and the lens-bag 22. Both the tension in the zonules 56 and the resulting mechanical disturbance of the lens-bag 22 can be also be used as indicators of the distance to the object-of-regard. In recognition of this, the rangefinder 18 can also include a tension measuring transducer in communication with the zonules 56 or a motion sensing transducer in communication with the lens-bag 22. These sensors can likewise be piezoelectric devices that generate a voltage in response to mechanical stimuli.

The activity of the rectus muscles 58 can also be used to infer the distance to an object-of-regard. For example, a contraction of the rectus muscles 58 that would cause the eye to converge medially can suggest that the object-of-regard is nearby, whereas contraction of the rectus muscles 58 that would cause the eye to gaze forward might suggest that the object-of-regard is distant. The rangefinder 18 can thus include a transducer that responds to either mechanical motion of the rectus muscles 58 or to the electrical activity that triggers that mechanical motion.

It is also known that when a person intends to focus on a nearby object, the iris 26 contracts the pupil 60. Another embodiment of the rangefinder 18 relies on this contraction to provide information indicative of the distance to the object-of-regard. In this embodiment, the rangefinder 18 includes a transducer, similar to that described above in connection with the rangefinder 18 that uses ciliary muscle or rectus muscle activity, to estimate the distance to the object-of-regard. Additionally, since contraction of the pupil 60 diminishes the light incident on the lens 12, the transducer 19 of the rangefinder 18 can include a photodetector for detecting this change in the light.

The foregoing embodiments of the rangefinder 18 are intended to be implanted into a patient, where they can be coupled to the anatomical structures of the eye. This configuration, in which the dynamic properties of one or more anatomical structures of the eye are used to infer the distance to an object-of-regard, is advantageous because those properties are under the patient's control. As a result, the patient can, to a certain extent, provide feedback to the rangefinder 18 by controlling those dynamic properties. For example, where the rangefinder 18 includes a transducer responsive to the ciliary muscle 54, the patient can control the index of refraction of the intraocular lens 12 by appropriately contracting or relaxing the ciliary muscle 54.

Other embodiments of the rangefinder 18 can provide an estimate of the range without relying on stimuli from anatomic structures of the eye. For example, a rangefinder 18 similar to that used in an auto-focus camera can be implanted. An example of such a rangefinder 18 is one that transmits a beam of infrared radiation, detects a reflected beam, and estimates range on the basis of that reflected beam. The output of the rangefinder 18 can then be communicated to the actuator 14. Since a rangefinder 18 of this type does not rely on stimuli from anatomic structures of the eye, it need not be implanted in the eye at all. Instead, it can be worn on an eyeglass frame or even hand-held and pointed at objects of regard. In such a case, the signal from the rangefinder 18 can be communicated to the actuator 14 either by a wire connected to an implanted actuator 14 or by a wireless link.

A rangefinder 18 that does not rely on stimuli from an anatomic structure within the eye no longer enjoys feedback from the patient. As a result, it is desirable to provide a feedback mechanism to enhance the range-finder's ability to achieve and maintain focus on an object-of-regard.

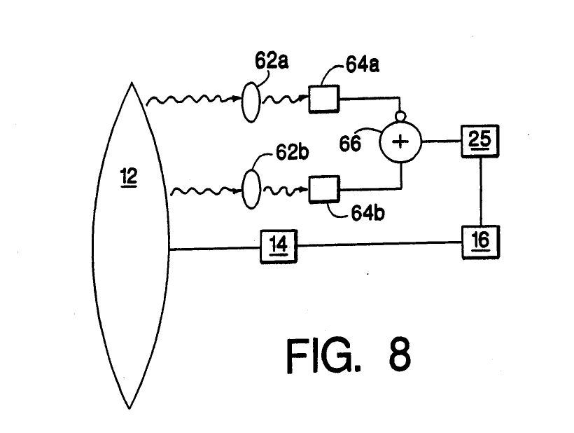

In a feedback mechanism as shown in FIG. 8, first and second lenslets 62a, 62b are disposed posterior to the intraocular lens 12. The first and second lenslets 62a, 62b are preferably disposed near the periphery of the intraocular lens 12 to avoid interfering with the patient's vision. A first photodetector 64a is disposed at a selected distance posterior to the first lenslet 62a, and a second photodetector 64b is disposed at the same selected distance posterior to the second lenslet 62b. The focal length of the first lenslet 62a is slightly greater than the selected distance, whereas the focal length of the second lenslet 62b is slightly less than the selected distance.

The outputs of the first and second photodetectors 64a, 64b are connected to a differencing element 66 that evaluates the difference between their output. This difference is provided to the digital signal processor 25. When the output of the differencing element 66 is zero, the intraocular lens 12 is in focus. When the output of the differencing element 66 is non-zero, the sign of the output identifies whether the focal length of the intraocular lens 12 needs to be increased or decreased, and the magnitude of the output determines the extent to which the focal length of the intraocular lens 12 needs to change to bring the lens 12 into focus. A feedback mechanism of this type is disclosed in U.S. Pat. No. 4,309,603, the contents of which are herein incorporated by reference.

In any of the above embodiments of the rangefinder 18, a manual control can also be provided to enable a patient to fine-tune the focusing signal. The digital signal processor 25 can then use any correction provided by the user to calibrate the range estimates provided by the rangefinder 18 so that the next time that that range estimate is received, the focusing signal provided by the digital signal processor 25 will no longer need fine-tuning by the patient. This results in a self-calibrating vision prosthesis 10.

The choice of which of the above range-finders is to be used depends on the particular application. For example, a lens 12 implanted in the posterior chamber 24 has ready access to the ciliary muscle 54 near the haptic 30 of the lens 12. Under these circumstances, a rangefinder that detects ciliary muscle activity is a suitable choice. A lens 12 implanted in the anterior chamber 34 is conveniently located relative to the iris 26 but cannot easily be coupled to the ciliary muscle 54. Hence, under these circumstances, a rangefinder that detects contraction of the iris 26 is a suitable choice. A lens 12 implanted in the cornea 36 is conveniently located relative to the rectus muscles 58. Hence, under these circumstances, a rangefinder that detects contraction of the rectus muscles 58 is a suitable choice. In the case of an aphakic patient, in which the natural lens 28 in the lens-bag 22 has been replaced by an intraocular lens 12, a rangefinder that detects zonule tension or mechanical disturbances of the lens-bag 22 is a suitable choice. In patients having a loss of function in any of the foregoing anatomical structures, a rangefinder that incorporates an automatic focusing system similar to that used in an autofocus camera is a suitable choice.

Power Source

As noted above, the controller 16, the rangefinder 18, and the actuator 14 shown in FIG. 1 require a power source 20. In one embodiment, the power source 20 can be an implanted battery 68. The battery 68 can be implanted in any convenient location, such as under the conjunctiva 70 in the Therron's capsule, or within the sclera. Unless it is rechargeable in situ, such a power source 20 will periodically require replacement.

In another embodiment, the power source 20 can be a photovoltaic cell 72 implanted in a portion of the eye that receives sufficient light to power the vision prosthesis 10. The photovoltaic cell 72 can be mounted on a peripheral portion of the lens 12 where it will receive adequate light without interfering excessively with vision. Alternatively, the photovoltaic cell 72 can be implanted within the cornea 36, where it will receive considerably more light. When implanted into the cornea 36, the photovoltaic cell 72 can take the form of an annulus or a portion of an annulus centered at the center of the cornea 36. This configuration avoids excessive interference with the patient's vision while providing sufficient area for collection of light.

Power generated by such a photovoltaic cell 72 can also be used to recharge a battery 68, thereby enabling the vision prosthesis 10 to operate under low-light conditions. The use of a photovoltaic cell as a power source 20 eliminates the need for the patient to undergo the invasive procedure of replacing an implanted battery 68.

The choice of a power source 20 depends in part on the relative locations of the components that are to be supplied with power and the ease with which connections can be made to those components. When the lens 12 is implanted in the cornea 36, for example, the associated electronics are likely to be accessible to a photovoltaic cell 72 also implanted in the cornea 36. In addition, a rechargeable subconjunctival battery 68 is also easily accessible to the photovoltaic cell 72. The disposition of one or more photovoltaic cells 72 in an annular region at the periphery of the cornea 36 maximizes the exposure of the photovoltaic cells 72 to ambient light.

When the lens 12 is implanted in the anterior chamber 34, one or more photovoltaic cells 72 are arranged in an annular region on the periphery of the lens 12. This reduces interference with the patient's vision while providing sufficient area for exposure to ambient light. For a lens 12 implanted in the anterior chamber 34, a rechargeable battery 68 implanted beneath the conjunctiva 70 continues to be conveniently located relative to the photovoltaic cells 72.

When the lens 12 is implanted in the posterior chamber 24, one or more photovoltaic cells 72 can be arranged in an annular region of the lens 12. However, in this case, the periphery of the lens 12 is often shaded by the iris 26 as it contracts to narrow the pupil 60. Because of this, photovoltaic cells 72 disposed around the periphery of the lens 12 may receive insufficient light to power the various other components of the vision prosthesis 10. As a result, it becomes preferable to dispose the photovoltaic cells 72 in an annular region having radius small enough to ensure adequate lighting but large enough to avoid excessive interference with the patient's vision.

Certain types of photovoltaic cells 72 are rechargeable by laser radiation. Such laser-rechargeable cells are useful because a laser can deliver considerable power to a small area. As a result, the photosensitive portions of such photovoltaic cells 72 can be made smaller than those of photovoltaic cells that are recharged by ambient light.

In some implementations, the photo-sensitive portion of the cell 72 is behind the iris 26. In these cases, the patient undergoes a brief recharging procedure in which the pupil 60 is dilated to expose the photo-sensitive portion for illumination by a laser. Since the laser delivers considerable power, the cell 72 can be fully charged within a recharging period that is short enough for the patient to endure.

In some photovoltaic cells 72, the photosensitive portion defines a spot near the periphery of the opening formed by the dilated pupil 60. In other photovoltaic cells 72, the photosensitive portion defines an annulus that encompasses the periphery of that opening. In either case, the charging laser beam has a profile corresponding to the shape of the photosensitive portion. Thus, in the latter case, the laser beam would have an annular power density profile.

Another power source 20 includes a thermoelectric battery, which develops a charge when exposed to a temperature differential. Such a battery exploits the natural temperature differential that exists in the eye. A thermoelectric battery 68 can be implanted within the iris 26, for example during an iridectomy. When thus implanted, the thermoelectric battery 68 exploits the temperature difference between the posterior chamber 24 and the slightly cooler anterior chamber 34. Alternatively, the thermoelectric battery 68 is implanted in the sclera to exploit the difference between the intra-ocular temperature and the subconjunctival temperature.

Another power source 20 includes a battery 68 that can be remotely charged by exposure to magnetic fields. In such prostheses 10, recharging is carried out without dilating the pupil 60 or requiring that the patient maintain any fixed posture or gaze. Instead, the patient relaxes in the vicinity of a suitable magnetic field.

In some power sources 20, the energy used to recharge the battery 68 can be captured by mechanical devices. For example, the same mechanism used to power a self-winding watch can be used to capture energy from the patient's daily eye or head movements. This captured energy recharges the battery 68. An appropriate self-winding movement can readily be fabricated using MEMS fabrication technology.

Alternatively, a mechanical linkage may be placed in mechanical communication with an anatomic structure of the eye, such as the ciliary body 54, the eyelid, or the eye muscle. Relative motion of any of these anatomic features relative to other anatomic features in the eye can then be used to provide sufficient power for recharging the battery 68.

One type of mechanical linkage is that provided by a dielectric elastomer. Such dielectric elastomers are known to develop a voltage in response to mechanical deformation. In these types of mechanical linkage, a dielectric elastomer is mechanically coupled to an anatomic structure of the eye, or to the eyelid. Mechanical energy associated with movement of these structures is thus captured by the compression or deformation of one or more dielectric elastomers. In some cases, the dielectric elastomers are arranged in an array of multilayer diaphragms. Examples of dielectric elastomer are disclosed in U.S. application Ser. No. 10/895,504, the contents of which are herein incorporated by reference.

Another type of power source 20 features a magnetic coupling between an anatomic structure of the eye and the lens 12. Suitable, anatomic structures include the ciliary body 54, the zonules 56, and sclera.

Such coupling is achieved by attaching magnets to the anatomic structure and the lens 12. As a result of this magnetic coupling, movement of the anatomic structure results in a corresponding movement, or deformation, of the lens 12.

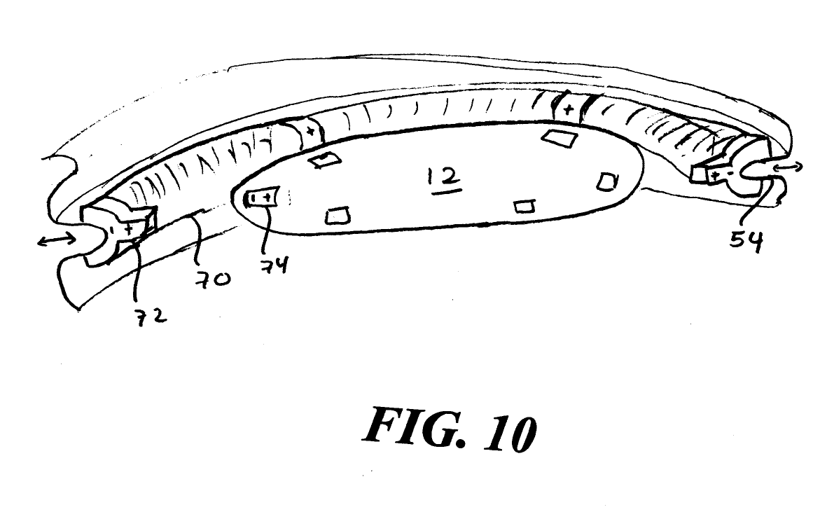

FIG. 10 shows one example in which a ring 70 is coupled to the ciliary body 54. Several ring-mounted magnets 72 are disposed circumferentially around the ring 70. Corresponding lens-mounted magnets 74 are disposed circumferentially around the lens 12. The lens-mounted magnets 74 and the ring-mounted magnets 72 are separated by a gap that is small enough to permit magnetic interaction between the ring-mounted magnets 72 and the lens-mounted magnets 74. As the ciliary body 54 contracts and relaxes, the spatial relationship between the lens-mounted magnets 74 and the ring-mounted magnets 72 changes. This change results in forces that shift the equilibrium position and shape of the lens 12. In this way, mechanical energy associated with an anatomic structure, in this case the ciliary muscle 54, is harnessed for changing the focus of the lens 12.

Extraocular Vision Prosthesis

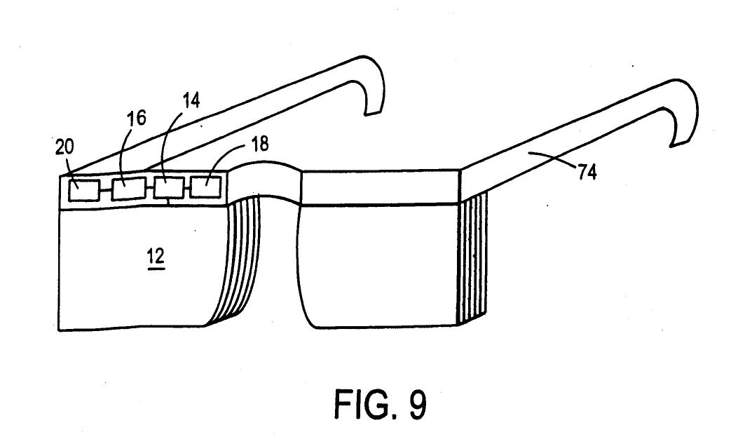

The lens 12 in FIG. 1 need not be an intraocular lens. In an alternative embodiment, shown in FIG. 9, the vision prosthesis 10, including the lens 12, is mounted on a frame 74 and worn in the manner of conventional eyeglasses. This embodiment largely eliminates those constraints on the size and location of the power source 20 that are imposed by the relative inaccessibility of the various anatomical structures of the eye as well as by the limited volume surrounding them.

In the embodiment shown in FIG. 9, the rangefinder 18 is typically of the type used in an autofocus camera together with the two-lenslet feedback mechanism described above in connection with the intraocular vision prosthesis 10. The lens 12, its associated actuator 14, and the power source 20 can be selected from any of the types already described above in connection with the intraocular embodiment of the vision prosthesis 10.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Having described the invention, and a preferred embodiment thereof, what I claim as new and secured by Letters Patent is:

1. A vision prosthesis comprising
an intraocular lens having refractive power that varies in response to a stimulus; and
an implantable power source for providing power to an actuator in communication with the lens for providing the stimulus.

2. The vision prosthesis of claim 1, further comprising the actuator.

3. The vision prosthesis of claim 1, wherein the intraocular lens has an index of refraction that varies in response to a stimulus.

4. The vision prosthesis of claim 1, wherein the intraocular lens has a shape that varies in response to a stimulus.

5. The vision prosthesis of claim 1, wherein the intraocular lens comprises lens elements that move relative to each other in response to a stimulus.

6. The vision prosthesis of claim 1, wherein the power source comprises a rechargeable power source.

7. The vision prosthesis of claim 6, wherein the power source comprises a photovoltaic cell.

8. The vision prosthesis of claim 7, wherein the photovoltaic cell comprises a light-receiving portion configured for disposition posterior to the iris.

9. The vision prosthesis of claim 8, wherein the light-receiving portion is annular.

10. The vision prosthesis of claim 7, wherein the photovoltaic cell is configured to be recharged by laser radiation.

11. The vision prosthesis of claim 6, wherein the rechargeable power source is configured to be recharged by exposure thereof to an electromagnetic field.

12. The vision prosthesis of claim 11, wherein the electromagnetic field comprises a magnetic field.

13. The vision prosthesis of claim 1, wherein the implantable power source comprises a thermoelectric cell.

14. The vision prosthesis of claim 6, further comprising means for capturing mechanical energy for recharging the power source.

15. The vision prosthesis of claim 14, wherein the means for capturing mechanical energy comprises means for capturing kinetic energy associated with movement of an anatomic structure.

16. The vision prosthesis of claim 6, wherein the implantable power source further comprises a self-winding mechanism configured to capture kinetic energy for recharging the rechargeable power source.

17. The vision prosthesis of claim 6, wherein the implantable power source further comprises a dielectric elastomer coupled to an anatomic structure of the eye for recharging the rechargeable power source.

18. The vision prosthesis of claim 1, wherein the implantable power source comprises a mechanical linkage configured for placement between an anatomic structure of the eye and the intraocular lens.

19. The vision prosthesis of claim 18, further comprising a magnet attached to the intraocular lens, the magnet being responsive to a force applied to the mechanical linkage.

20. The vision prosthesis of claim 19, wherein the mechanical linkage comprises a ring configured for attachment to the ciliary body, and a magnet attached to the ring for exerting force on the magnet attached to the intraocular lens.
